(19) United States
(12) Patent Application Publication
(10) Pub. No.: US 2017/0239067 A1
(43) Pub. Date: Aug. 24, 2017

(54) Radiopaque Trial Disc, Plate, and Method of Use

(71) Applicant: ECA Medical Instruments, Newbury Park, CA (US)

(72) Inventor: John Nino, Simi Valley, CA (US)

(73) Assignee: ECA Medical Instruments, Newbury Park, CA (US)

(21) Appl. No.: 15/588,375

(22) Filed: May 5, 2017

Related U.S. Application Data
(63) Continuation of application No. PCT/US2015/059290, filed on Nov. 5, 2015.
(60) Provisional application No. 62/075,717, filed on Nov. 5, 2014.

(51) Int. Cl. A61F 2/46; A61F 2/44

(52) U.S. Cl. CPC A61F 2/4684; A61F 2/4611; A61F 2/442; A61F 2002/3008

(57) ABSTRACT

Surgical kits comprising a tool having a handle and a shaft affixed to the handle at one end and a free end having a threaded connection, at least two disc inserts, each disc insert comprising a rounded square having an annular wall surrounding a top surface and a bottom surface, wherein a threaded catch is formed in each disc insert to mate with the threaded connection, and wherein the interface between the disc insert top surface and annular wall forms a first perimeter and the interface between the disc insert bottom surface and the annular wall forms a second perimeter. At least a portion of the disc inserts can be radiopaque and a portion can be radiolucent. Also provided are kits having one or more disc inserts and one or more trial plates, each trial plate comprising a plurality of positioning guides.

### CROSS REFERENCE

This application claims priority to U.S. Provisional Patent Application 62/075,717 filed Nov. 5, 2014, the contents of which are incorporated herein by reference as if fully set forth herein.

### BACKGROUND

1. Field

This disclosure relates to a kit of disposable devices for assessment of positioning of spinal implant and or fusion devices.

2. General Background

Spinal fusion requires the surgeon to remove diseased or damaged disc body and utilize spacer or inserts in combination with plates, screws, cages and the like to affix adjacent vertebra together.

### DISCLOSURE

Briefly stated, kits providing swappable disposable radiopaque spinal disc spacers or inserts of various sizes and an instrument to insert and remove, “swap”, the spacers are disclosed. The inserts may be partially radiolucent and partially radiopaque to facilitate visualization under X-rays/fluoroscope. The trial plate may be partially radiolucent and partially radiopaque to facilitate visualization under X-rays/fluoroscope. At least two inserts of varying sizes are supplied with a kit.

Aspects of implementations include kits comprising a tool having a handle and a shaft affixed to the handle at one end and a free end having a threaded connection, the kits further comprising at least two disc inserts, each disc insert comprising a rounded square having an annular wall surrounding a top surface and a bottom surface, wherein a threaded catch is formed in each disc insert to mate with the threaded connection, and wherein the interface between the disc insert top surface and annular wall forms a first perimeter and the interface between the disc insert bottom surface and the annular wall forms a second perimeter. In some instances at least a portion of a disc insert is radiopaque. In some instances at least a portion of the disc inserts is radiopaque and a portion is radiolucent.

Aspects of implementations include kits comprising one or more disc inserts, each disc insert comprising a rounded square having an annular wall surrounding a top surface and a bottom surface, wherein the interface between the disc insert top surface and annular wall forms a first perimeter and the interface between the disc insert bottom surface and the annular wall forms a second perimeter, and wherein the disc insert is at least partially radiopaque, and the kits further comprise one or more trial plates, each trial plate comprising a plurality of positioning guides. In some instances at least a portion of a trial plate is radiopaque. In some instances at least a portion of a trial plate is radiopaque and a portion is radiolucent. In some instances one of the trial plates and one of the inserts is more radiopaque than the other.

### DRAWINGS

The above-mentioned features of the present disclosure will become more apparent with reference to the following description taken in conjunction with the accompanying drawings wherein like reference numerals denote like elements and in which:

FIGS. 1A-1C are disc insert and tool;

FIG. 2 shows a tool inserting a disc insert;

FIG. 3 shows a disc insert between vertebra

FIG. 4 shows a trial plate and disc insert; and,

FIG. 5A-5C shows aspects of radiopaque or marked trial plates and an insert.

As shall be appreciated by those having ordinary skill in the art, the figures are not to scale, and modifications to scale within a figure or across the figures are considered within the present disclosure.

### FURTHER DESCRIPTION

Being disposable and single use the kit parts described herein avoid exposure of patients to radiopaque materials used herewith for all but a brief exposure. Further, the coating on coated embodiments may be extremely thin, to again limit the amount of such materials used or exposed to humans, due to the disposable nature of the kit parts.

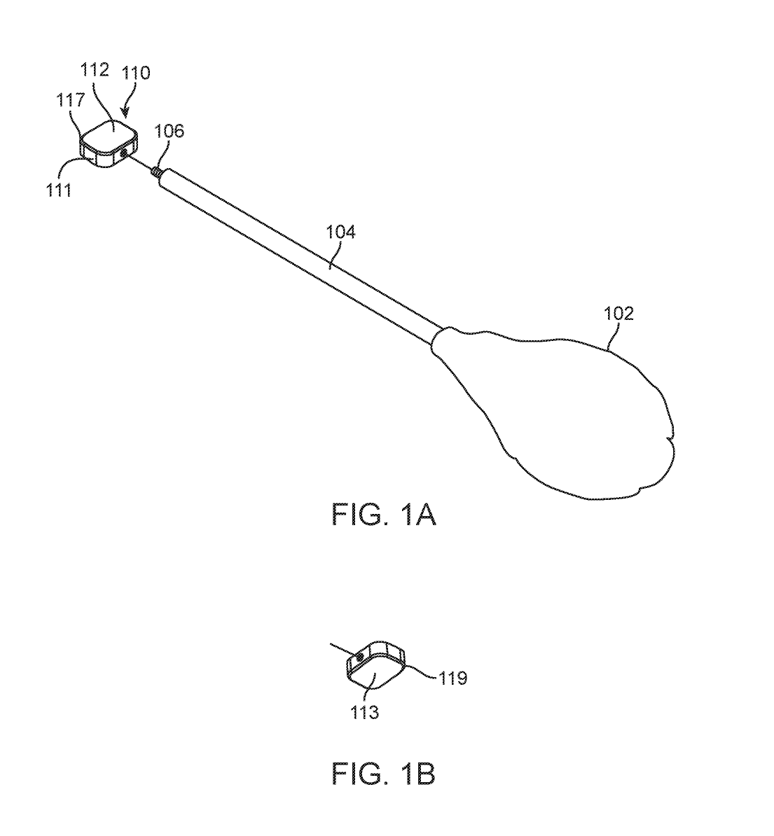

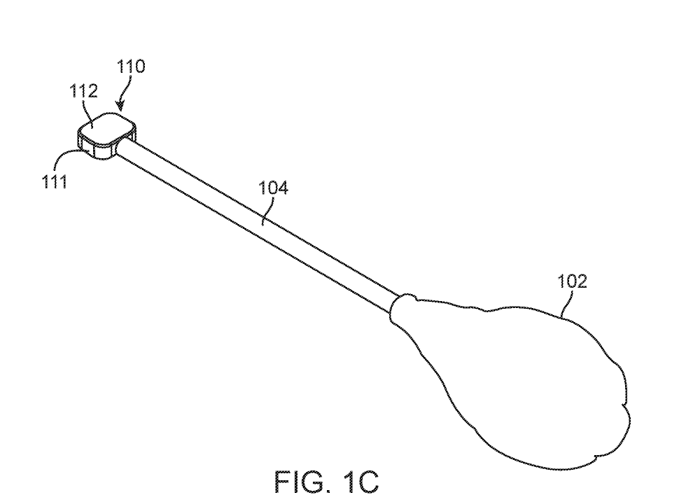

FIGS. 1A-1C show a tool 100 is disclosed having a handle 102 for grasping and a shaft 104 affixed to the handle at one end and a free end 105 having a threaded connection 106. An insert 110 is disclosed of a size to approximate an intervertebral space. A kit may contain a plurality of inserts of varying sizes to give a surgeon choice in selecting the insert which best approximates an intervertebral space. The insert 110 is roughly a rounded square having an annular wall 111 surrounding a top surface 112 and a bottom surface 113. A threaded catch 115 is formed in the insert to mate with the threaded connection 106. The interface between the disc insert top surface and annular wall 111 forms a first perimeter 117 and the interface between the disc insert bottom surface and the annular wall 111 forms a second perimeter 119.

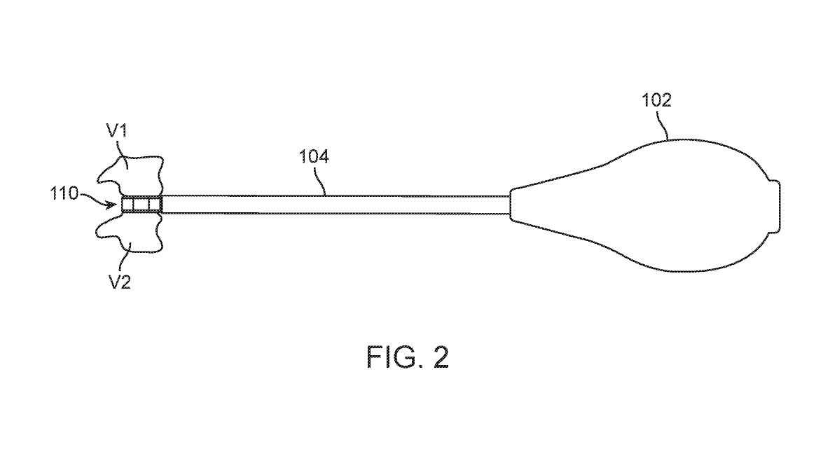

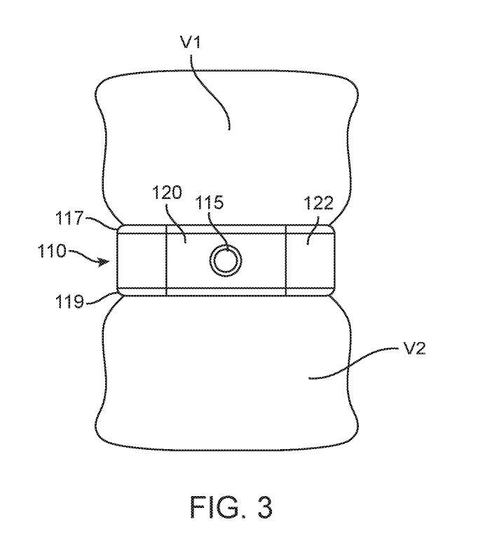

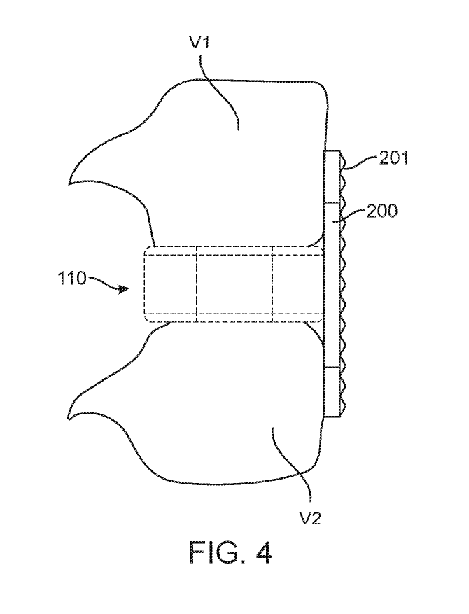

FIGS. 1A-5C show aspects of exemplary implementations of devices, methods and system which may include a kit with inserts 110 of various sizes. The inserts can be formed of plastic having radiopaque material therein. The insert may be coated with a radiopaque material or marker on one or more of a top surface 112, a bottom surface 113, an annular wall 111 and a perimeter 117/119. In some instances the insert may have radiolucent regions 120 or portions and radiopaque portions or regions 122.

When the tool 100 is connected to the insert 110, the insert is then inserted between a top vertebra “V1” and a bottom vertebra “V2”.

A system and method of use includes a kit with various size and thickness inserts 110 each connectable to the tool. The surgeon assesses the physical space for an insert and tries various inserts to ascertain the right size for an implantable device. After assessing the size and height of the insert, which can include visualization of the insert between an upper vertebra and a lower vertebra via at least one radiopaque region 122, radiopaque coating or marker. After the size and shape for the spinal final spinal disc spacer device (not shown) is assessed the insert is removed and replaced with a disc insert (not shown).

The insert 110 may have perimeters 117/119 and either perimeter, or a portion thereof, may be formed of or coated with a radiopaque material.

A trial plate 200 also disclosed. A trial plate mimics the size and shape of a plate, cage or other device used in spinal fusion procedures. Part of the kit may include different sized trial plates. The trial plate may be coated 201 with a radiopaque material.

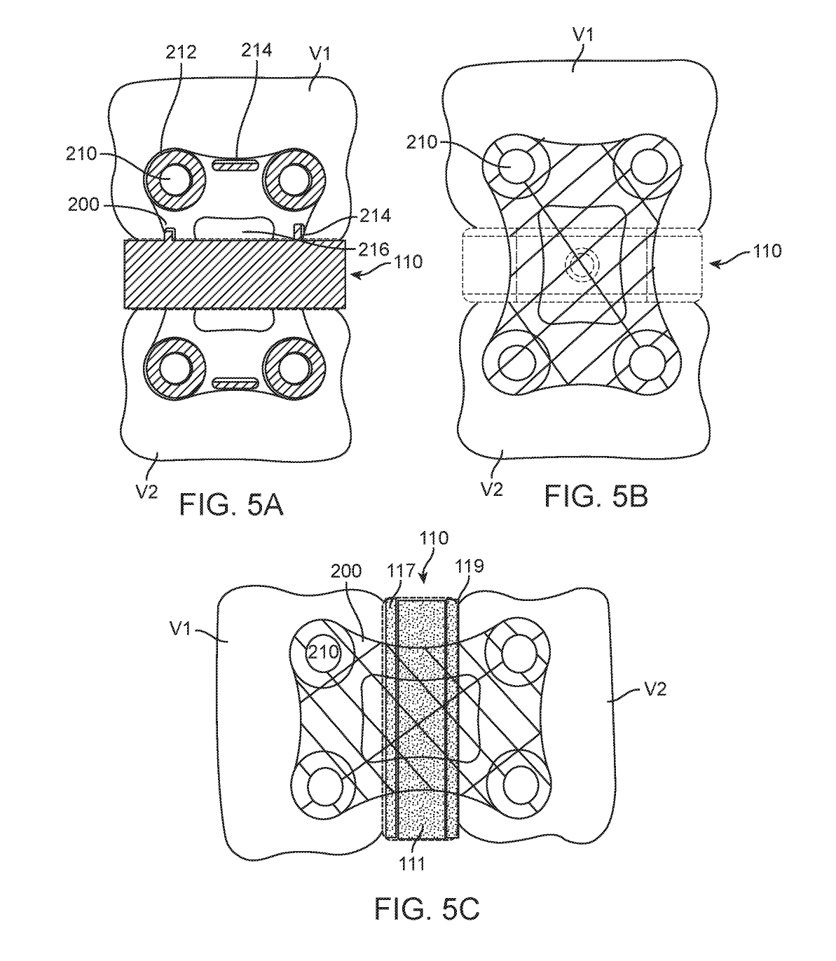

FIGS. 5A-5C show aspects of trial plate and insert combinations with varying radiopaque portions. Trial plates provide positioning guides 210 for the placement of drills, drill guides or fasteners (such as screws) for affixing a stabilizing device to vertebra during spinal fusion procedures. However, vertebra size and shape vary and a kit having multiple plates and/or inserts to choose from allows a surgeon to customize the fit to the patient. In some instance radiopaque guide regions 212 surround positioning guides 210. In some instances radiopaque edge markers 214 are formed at the edges of the trial plate. In some instance radiolucent regions 216 are also provided on the trial plate. In FIG. 5A the insert 110 is radiopaque and visible through the radiolucent portion 216 of the trial plate 200.

In FIG. 5B the trial plate is radiopaque except for the positioning guides 210. In FIG. 5C the trial plate 200 is radiopaque, but less radiopaque then the insert 110 or portions thereof. By selection of concentrations of the radiopaque materials the insert or trial plate may be made distinguishable from the other. In some implementations, suitable materials for the insert or trial plate may include thermocomp (compound EX06430H from material manufacturer: Sabic Innovative Plastics, 1 Plastics Avenue Pittsfield, Mass. 01201), which may include barium sulfate and ULTEM™ polyetherimide resin; other suitable materials can include tantalum, gold, platinum, iridium, palladium, and rhodium and are well recognized for their biocompatibility. Radiopaque coatings can be applied to plastics using a high-vacuum deposition process that results in dense thin—film coatings (typically less than 10 μm) that adhere to the surface of the device. One or more of Barium Sulfate (BaSO₄), Bismuth Subcarbonate (Bi₂O₂(CO₃)), Bismuth Oxychloride (BiOCl), Bismuth Trioxide (Bi₂O₃), and Tungsten (W) may be used to provide radiopacity to one or more inserts or trial plates in some implementations. Radiopaque materials may be combined with and mixed into materials used for injection molding and thereby distributed throughout at least a portion of a device so manufactured.

To make the insert or trial plate distinguishable from the other the perimeter 117/119 may be radiopaque and the annular wall 11 not, or vice versa. Or the perimeters may be more radiopaque than the trial plate 200 or portions thereof.

While the method and agent have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular implementation, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A kit comprising:
a tool (100) having a handle (102) and a shaft (104) affixed to the handle at one end and a free end (105) having a threaded connection (106);
at least two disc inserts (110), each disc insert comprising:
a rounded square having an annular wall (111) surrounding a top surface (112) and a bottom surface (113),
wherein a threaded catch (115) is formed in each disc insert to mate with the threaded connection (106), and
wherein the interface between the disc insert top surface and annular wall (111) forms a first perimeter (117) and the interface between the disc insert bottom surface and the annular wall (111) forms a second perimeter (119).

2. The kit of claim 1 wherein at least a portion of each of the at least two disc inserts is radiopaque.

3. The kit of claim 1 wherein at least a portion of each of the at least two disc inserts is radiopaque and at least a portion of each of the at least two disc inserts is radiolucent.

4. The kit of claim 1 wherein each of the at least two disc inserts is formed of plastic having radiopaque material therein.

5. The kit of claim 1 wherein each of the at least two disc inserts is coated with a radiopaque material or marker on one or more of the top surface (112), the bottom surface (113), the annular wall (111), the first perimeter (117), and the second perimeter (119).

6. The kit of claim 1 wherein at least two of the disc inserts have different size, shape, or both relative to each other.

7. A kit comprising:
one or more disc inserts, each disc insert comprising:
a rounded square having an annular wall (111) surrounding a top surface (112) and a bottom surface (113),
wherein the interface between the disc insert top surface and annular wall (111) forms a first perimeter (117) and the interface between the disc insert bottom surface and the annular wall (111) forms a second perimeter (119), and
wherein the disc insert is at least partially radiopaque, and
one or more trial plates (200), each trial plate comprising a plurality of positioning guides (210).

8. The kit of claim 7 wherein the one or more trial plates is radiopaque.

9. The kit of claim 7 wherein at least a portion of the one or more trial plates is radiopaque.

10. The kit of claim 7 wherein at least a portion of the one or more trial plates is radiopaque and at least a portion of the one or more trial plates is radiolucent.

11. The kit of claim 7 wherein one of the one or more trial plates and one of the one or more disc inserts is more radiopaque than the other.

12. The kit of claim 7 wherein at least one trial plate comprises radiopaque guide regions (212) surrounding the plurality of positioning guides (210).

13. The kit of claim 7 wherein at least one trial plate comprises radiopaque edge markers (214) formed at the edges of the trial plate.

14. The kit of claim 7 wherein at least one trial plate comprises at least one radiolucent region (216).

15. The kit of claim 7 wherein the first perimeter, second perimeter, or both perimeters of at least one disc insert is more radiopaque than at least one trial plate (200).
